(19) United States
(12) Patent Application Publication
(10) Pub. No.: US 2019/0021289 A1
(43) Pub. Date: Jan. 24, 2019

(54) SEPARATOR FOR FEMALE AND MALE MOSQUITO PUPAE

(71) Applicant: GUANGZHOU WOLBAKI BIOTECH CO., LTD, Luogang District Guangzhou, Guangdong (CN)

(72) Inventors: Xiaohua WANG, Luogang District Guangzhou, Guangdong (CN); Jian ZHU, Luogang District Guangzhou, Guangdong (CN); Zhiyong XI, Luogang District Guangzhou, Guangdong (CN); Jixin ZHANG, Luogang District Guangzhou, Guangdong (CN); Yongping LUO, Luogang District Guangzhou, Guangdong (CN)

(21) Appl. No.: 16/068,337

(22) PCT Filed: Jun. 1, 2016
(86) PCT No.: PCT/CN2016/084269

§ 371 Date: Jul. 5, 2018

(30) Foreign Application Priority Data
Jan. 6, 2016 (CN) 201610014021.8

(51) Int. Cl. A01K 29/00; A01K 67/033; B07B 13/04

(52) U.S. Cl. CPC A01K 29/00; B07B 13/04; A01K 67/033

(57) ABSTRACT

The present disclosure discloses a separator of male and female mosquito pupae, comprising a fixed plate (2) configured on an inclined baseplate (1), an adjusting plate (3) spaced from the fixed plate (2), and a guide plate (8), wherein an interval between the adjusting plate (3) and the fixed plate (2) tapers from top to bottom, to a interval exit, and interval adjustments are configured go through the adjusting plate (3) from upper part and lower part of the fixed plate (2). The guide plate (8) is configured below the interval exit. The structure of the present disclosure is simple and reasonable, the mosquito larva, male pupae and female pupae can be separated and collected in sequence according to their different chest width. The operation is simple, the separation speed is fast, and the ratio of separating male pupae can reach over 99%, being a good separation effect.

### CROSS-REFERENCE TO RELATED APPLICATIONS

This application claims priority to PCT Application No. PCT/CN2016/084269, having a filing date of Jun. 1, 2016, based on CN 201610014021.8, having a filing date of Jan. 6, 2016, the entire contents both of which are hereby incorporated by reference.

### TECHNICAL FIELD

The following relates to a technical field of sterile male insect releasing, in particular to a separator of male and female mosquito pupae.

### BACKGROUND

In the field of pest control, sterile male insect releasing is an emerging technique, i.e. a sterile male insect is bred, then released, such that male and female insect cannot breed their progeny, in order to eliminate pest.

In large-scale scheme of male mosquito releasing, male mosquito pupae must be separated from a mass of mixed mosquito pupae, the smaller the proportion of female pupae therein, the better the release effect. Therefore, it is desirable to have a separator that separates male mosquito pupae from the mixed pupae in simple and effective manner.

### SUMMARY OF THE INVENTION

The present disclosure is directed to the problems existing in the prior art as described above, and provides a separator of male and female mosquito pupae for separating bred male pupae from mixed female and male pupae.

Technical solution of the present disclosure is as follows:

A separator of male and female mosquito pupae, comprises a fixed plate configured on an inclined baseplate, an adjusting plate spaced from the fixed plate, and a guide plate. An interval between the adjusting plate and the fixed plate tapers from top to bottom, to an interval exit, and the interval adjustments are configured go through the adjusting plate from upper part and lower part of the fixed plate. The guide plate is configured below the interval exit.

Utilizing the above technical solutions, the interval between the adjusting plate and the fixed plate is adjusted be the adjustments, when the mosquito pupae, mosquito larva and certain amount of water are poured in the top opening of interval between the fixed plate and the adjusting plate, the mosquito pupae group slide down along the opposite surfaces of the adjusting plate and fixed plate with the water flows, only the larva and pupae meet the separation requirements can pass through the interval and reach the guide plate, and the larger one will remain. Therefore, the larva, the female pupae and the male pupae is separated according to the chest width.

The separator of male and female mosquito pupae further comprises a holder, a movable bracket provided on the top of the holder and fixed on the baseplate, a fixed bracket provided on the lower part of the baseplate, and an adjusting lever provided on the fixed bracket, the adjusting lever is movably connected to the holder in order to adjust the angle between the baseplate and the holder.

Utilizing the movable bracket and the angle adjusting lever to regulate the inclined angle of the baseplate, thereby adjusting the inclined angle between the fixed plate and the adjusting plate, so that the water flow is smooth and to separation is thorough, and the operator can observe the separation process at a proper angle.

The interval adjustments are bolts through the adjusting plate from the fixed plate, a compression spring is sleeved on the bolt between the adjusting plate and the fixed plate such that two ends of the spring abut against the opposite surfaces of the fixed plate and the adjusting plate. The fixed plate and the adjusting plate can be positioned by the bolt and be tensioned by the compression spring to eliminate the clearance generated due to a loose fitting, thereby effectively preventing the shaking and ensuring the accuracy of the separation.

A baffle plate bended outward is provided on the top of the adjusting plate, such that the mosquito pupae group and water can be poured into the interval without splashing out.

Along the flow of the mosquito pupae, the mosquito pupae flow to lower side of the guide plate, the lower side of the guide plate forms a collect port. Left and right sides of the guide plate are upward flanged and make the collect port narrower than the upper side of the guide plate. The separating mosquito pupae is prevented from leaking or flushing by the flange part, therefore, the separating mosquito pupae will be collected into the collect port maximally.

The present disclosure can separate and collect the mosquito larva, male pupae and female pupae in sequence according to their different chest width. The operation is simple, the separation speed is fast, and the ratio of separating male pupae can reach over 99%, being a good separation effect.

### BRIEF DESCRIPTION OF DRAWINGS

FIG. 1 is a structural schematic view of the present invention;

FIG. 2 is a side view of FIG. 1;

### REFERENCE NUMERALS

1 baseplate
2 fixed plate
3 adjusting plate
4 fixed bracket
5 adjusting lever
6 movable bracket
7 holder
8 guide plate
9 baffle plate
10 bolt
11 knob
12 compression spring

### DETAILED DESCRIPTION OF PREFERRED EMBODIMENTS

The present disclosure will be described further in details hereinafter with reference to the following embodiment and figures, to make the above objects, features, and advantages of the present disclosure more apparent and comprehensible.

### Embodiment

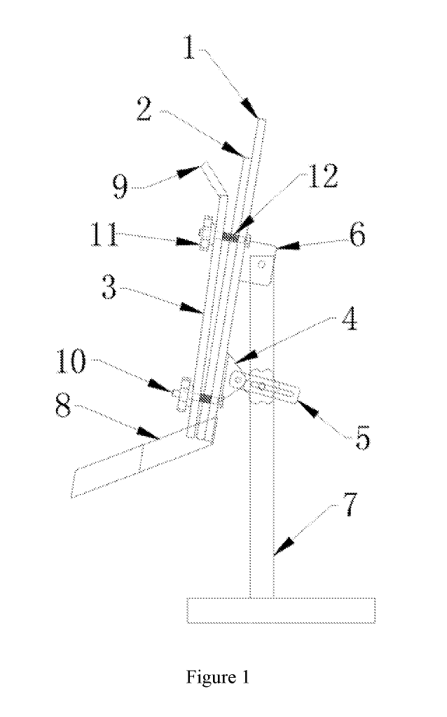

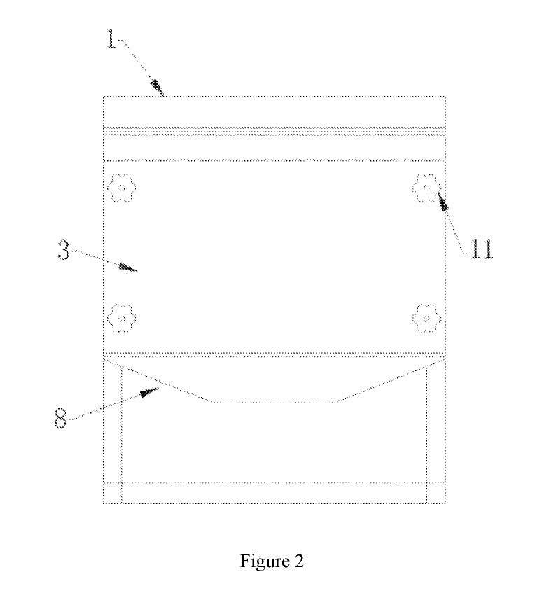

As shown in FIG. 1 and FIG. 2, a separator of male and female mosquito pupae, comprises a fixed plate 2 configured on an inclined baseplate 1, an adjusting plate 3 spaced from the fixed plate 2, and a guide plate. An interval between the adjusting plate 3 and the fixed plate 2 tapers from top to bottom, to an interval exit, and the interval adjustments are configured go through the adjusting plate 3 from upper part and lower part of the fixed plate 2. The guide plate 8 is configured below the interval exit.

The separator of male and female mosquito pupae further comprises a holder 7, a movable bracket 6 provided on the top of the holder 7 and fixed on the baseplate 1, a fixed bracket 4 provided on the lower part of the baseplate 1, and an adjusting lever 5 provided on the fixed bracket 4, the adjusting lever 5 is movably connected to the holder 7 in order to adjust the angle between the baseplate 1 and the holder 7. The interval adjustments are bolts 10 through the adjusting plate 3 from the fixed plate 2, a compression spring 12 is sleeved on the bolt 10 between the adjusting plate 3 and the fixed plate 2 such that two ends of the spring 12 abut against the opposite surfaces of the fixed plate 2 and the adjusting plate 3. A baffle plate 9 bended outward is provided on the top of the adjusting plate 3. Along the flow of the mosquito pupae, the mosquito pupae flow to lower side of the guide plate 8, the lower side of the guide plate 8 forms a collect port. Left and right sides of the guide plate 8 are upward flanged and make the collect port narrower than the upper side of the guide plate 8.

In practice, the interval adjustment may be a bolt 10, the upper part of the adjusting plate 3 is provided with two bolts 10 near both sides of the adjusting plate 3 respectively, the end of the bolt 10 exposed above the adjusting plate 3 is provided with an adjusting knob 11, the other end of the bolt 10 goes through the fixed plate 2 until the baseplate 1. Likewise, the lower part of the adjusting plate 3 is provided with two bolts 10 near both sides of the adjusting plate 3 respectively. Utilizing the above technical solutions, the interval between the adjusting plate 3 and the upper part of the fixed plate 2 can be adjusted properly by the interval adjustment according to the chest width of the mosquito larva, the female mosquito pupa and the male mosquito pupa such that the mosquito pupae, which need to be separated, and certain amount of water can be poured in the top opening of interval between the fixed plate 2 and the adjusting plate 3, while the interval between the adjusting plate 3 and the lower part of the fixed plate 2 is regulated narrow enough by the interval adjustment such that only the mosquito larva with minimum chest width can pass through the slot exit, without the female mosquito pupa and the male mosquito pupa. Therefore, when the mosquito pupae, mosquito larva and certain amount of water are poured in the interval, the mosquito larva will be separated to the guide plate 8. Again, the interval between the adjusting plate 3 and the lower part of the fixed plate 2 is regulated wider such that only the male pupae can pass through the slot exit, without the female mosquito pupa such that the male pupae are separated and the female pupae will stay on the fixed plate 2 or the adjusting plate 3. Therefore, the larva, the female pupae and the male pupae can be separated.

The structure of the present disclosure is simple and reasonable, the mosquito larva, male pupae and female pupae can be separated and collected in sequence according to their different chest width. The operation is simple, the separation speed is fast, and the ratio of separating male pupae can reach over 99%, being a good separation effect.

The foregoing is merely the specific embodiments of the present disclosure, and is not intended to limit the protection scope of the present disclosure. Any replacement or improvement made without departing from the concept of the present disclosure is within the protection scope of the present disclosure.

1. A separator of male and female mosquito pupae, comprising a fixed plate configured on an inclined baseplate, an adjusting plate spaced from the fixed plate, and a guide plate, wherein an interval between the adjusting plate and the fixed plate tapers from top to bottom, to an interval exit, and interval adjustments configured on upper part and lower part, of the fixed plate and the adjusting plate; the guide plate is configured below the interval exit.

2. The separator of male and female mosquito pupae of claim 1, further comprising a holder, a movable bracket provided on the top of the holder and fixed on the baseplate, a fixed bracket provided on the lower part of the baseplate, and an adjusting lever provided on the fixed bracket, the adjusting lever is movably connected to the holder in order to adjust the angle between the baseplate and the holder.

3. The separator of male and female mosquito pupae of claim 2, wherein the interval adjustment may be bolts through the adjusting plate from the fixed plate, a compression spring is sleeved on the bolt between the adjusting plate and the fixed plate such that two ends of the spring abut against the opposite surfaces of the fixed plate and the adjusting plate.

4. The separator of male and female mosquito pupae of claim 1, wherein a baffle plate bended outward is provided on the top of the adjusting plate.

5. The separator of male and female mosquito pupae of claim 1, wherein along the flow of the mosquito pupae, the mosquito pupae flow to lower side of the guide plate, the lower side of the guide plate forms a collect port, left and right sides of the guide plate are upward flanged and make the collect port narrower than the upper side of the guide plate.

6. The separator of male and female mosquito pupae of claim 2, wherein a baffle plate bended outward is provided on the top of the adjusting plate.

7. The separator of male and female mosquito pupae of claim 3, wherein a baffle plate bended outward is provided on the top of the adjusting plate.

8. The separator of male and female mosquito pupae of claim 2, wherein along the flow of the mosquito pupae, the mosquito pupae flow to lower side of the guide plate, the lower side of the guide plate forms a collect port, left and right sides of the guide plat are upward flanged and make the collect port narrower than the upper side of the guide plate.

9. The separator of male and female mosquito pupae of claim 3, wherein along the flow of the mosquito pupae, the mosquito pupae flow to lower side of the guide plate, the lower side of the guide plate forms a collect port, left and right sides of the guide plat are upward flanged and make the collect port narrower than the upper side of the guide plate.
